(19) United States
(12) Patent Application Publication
(10) Pub. No.: US 2007/0162002 A1
(43) Pub. Date: Jul. 12, 2007

(54) Device for stabilizing the spine

(76) Inventor: Alain Tornier, Saint Ismier (FR)

Correspondence Address: Ralph A. Dowell of DOWELL & DOWELL P.C., 2111 Eisenhower Ave, Suite 406, Alexandria, VA, 22314, US

(21) Appl. No.: 11/634,374

(22) Filed: Dec. 6, 2006

Related U.S. Application Data
(60) Provisional application No. 60/748,580, filed on Dec. 9, 2005.

(30) Foreign Application Priority Data
Dec. 7, 2005 (FR) 05 12428

(51) Int. Cl. A61F 2/30

(52) U.S. Cl. 606/61

(57) ABSTRACT

This device comprises two vertebral assemblies designed to be fixed respectively to the bone of two different vertebrae. In order to guide the vertebrae effectively and in a stable manner for reproducing an intervertebral articular connection, rigid means connect the two vertebral assemblies to one another and are designed such that, when the device is in implantation configuration, they can be connected to each assembly so as to slide along a relative guide trajectory which, projected in the sagittal plane of the spine, is curved along the spine, having a concavity directed toward the spine and being centered at a zone contained in the interosseous space delimited between the two vertebrae.

The present invention relates to a device for dynamically stabilizing the spine, designed to be implanted along the vertebral column with a view to stabilizing at least two vertebrae relative to one another, while reproducing an intervertebral articular connection. Such stabilization is sought especially in the context of treatment of the degenerative or injured spine. The invention more particularly concerns the treatment of the dorsolumbar spine, but applies also to treatment of the cervical spine.

To treat an intervertebral instability, a first known possibility lies in fusing two adjacent vertebrae, which amounts to depriving these two vertebrae of their freedom of relative movement. For this purpose, totally rigid assemblies are implanted in a fixed manner along the spine, in order to permanently block the articular connection between the two vertebrae that are to be fused. An example of such an assembly, with a completely immobile structure, is disclosed in U.S. Pat. No. B-6,328,738. However, this arthrodesis procedure leads to degeneration of the adjacent disks, and the latter then have to be treated at a later stage.

US-A-2004/158,250 discloses also an assembly intended to be used to fuse two adjacent vertebrae. This assembly comprises two plate members that are fixed to two vertebral bodies and that are, just after their fixation, linked by a straight sliding mechanical linking. This mobility is also very temporary, because the assembly is quickly immobilized in its whole due to the settling of the space between the vertebral bodies by a graft, being noted the fact that this graft risks to be initially excessively compressed by both plate members.

Another known possibility for treating the spine involves intervening at an earlier stage than for arthrodesis and entails implantation of a dynamic stabilization device, as proposed in WO-A-03/094699, for example. To this end, the device comprises, on the one hand, bone-anchoring screws anchored in two immediately adjacent vertebrae, in the area of their pedicle, and, on the other hand, elastic elements for connection between these screws. These flexible elements, joined rigidly to each screw, relieve the intervertebral disk and correct any excess pressure in the area of the articular surfaces between this disk and the vertebrae. These devices provide greater patient comfort, because they allow the mobility of the spine to be retained. In practice, however, the use of these devices connecting the vertebrae in a flexible manner proves awkward: it is difficult to gauge the flexibility of the connection elements, since this has to be adapted to each patient depending on the disease and the morphology, and, in the long term, there is a risk of the elastic behavior of these elements changing. The fact that these parameters are difficult to control means that it is not possible to guarantee complying with the kinematics of the spine, and this may lead to poor stabilization of the intervertebral distance and to aggravation of the damage that it is sought to treat.

The object of the present invention is to make available a device for dynamically stabilizing the spine that more faithfully reproduces the anatomical movement of the vertebrae, is more effective in stabilizing the treated vertebrae and is more reliable over the course of time.

To this end, the invention relates to a device for dynamically stabilizing the spine intended to reproduce an intervertebral articular connection, comprising at least two vertebral assemblies designed to be each fixed respectively to the bone of a vertebra from among at least two different vertebrae of the spine, this device additionally comprising rigid means for connection between the two vertebral assemblies or between two of the vertebral assemblies, characterized in that the rigid means and the vertebral assemblies are designed such that, when the device is in implantation configuration, they are adapted to be connected to one another so as to slide along a relative guide trajectory which, projected in the sagittal plane of the spine, is curved along the spine, having a concavity directed toward the spine and being centered at a zone contained within of the interosseous space delimited between the two vertebrae to which the two assemblies are fixed.

The term “implantation configuration” is understood as the configuration in which the device is completely implanted in the vertebrae of the spine, in other words after the end of the surgical intervention for implanting this device. This implantation configuration thus corresponds to the postoperative configuration of the device, after consolidation of the vertebrae provided with this device.

The use of the rigid connection means for connecting the vertebral assemblies makes it possible to give the device a kinematic behavior that is stable over the course of time. By virtue of the sliding arrangement obtained, these rigid means provide the vertebral assemblies with predetermined and reliable guide trajectories, guaranteeing that the intervertebral articular movements are effectively centered at one or more predetermined intervertebral zones, so that the behavior is almost identical to or, at the very least, as close as possible to the normal anatomical behavior of the spine. By that way, in use, the intervertebral space is maintained, that-is-to say that this space is not reduced, nor even compressed by the dynamic action of the device, because the latter takes in charge the stresses related to the movements of the spine. Moreover, the implantation of the device according to the invention proves easy, since the internal mobility of the device lies essentially, or even exclusively, in the area of the sliding connections between the vertebral assemblies and the mechanical means connecting them.

According to an advantageous embodiment of the invention, the vertebral assemblies are respectively designed to be fixed to two adjacent vertebrae, and the projection, in the sagittal plane of the spine, of the relative guide trajectory, between the connection means and each of the two associated vertebral assemblies, is centered at a zone contained within the disk space separating the two vertebrae to which the two assemblies are fixed. In this case, the device according to the invention in fact stabilizes two immediately adjacent vertebrae, while guaranteeing them a certain mobility, essentially in terms of flexion and extension, centered on the intervertebral disk space, that is to say a freedom of movement close to the normal anatomical freedom. Indeed, the device supports the main part, and even the totality, of the stresses applying on the intervertebral disk, leaving its mobility to this disk.

Advantageously, each vertebral assembly comprises two subassemblies that can be fixed to one and the same vertebra, on either side of its spinous process.

According to a particularly simple and effective structure of the device according to the invention, the connection means for the two assemblies comprise two inwardly curved rigid rails for guiding the vertebral assemblies, which rails are substantially parallel to one another and along which, respectively, opposite lateral parts of each vertebral assembly are designed to slide along the aforementioned guide trajectory when the device is in the implantation configuration.

According to other advantageous characteristics of this device, taken in isolation or in all of the technically possible combinations:
the two rails are designed to extend along and on either side of the spinous processes of the vertebrae;
the two rails are supported by one and the same component designed to extend, in the longitudinal direction of the rails, along the anterior side of the vertebrae;
each lateral part of each vertebral assembly comprises a head for sliding along the corresponding rail, this head being equipped with a stud received in a guide orifice delimited by the rail;
each lateral part of each vertebral assembly comprises a pedicle-anchoring rod or a clip for fastening on the process;
the longitudinal direction of the stud of each head is adjustable relative to the rod or to the clip before the device is brought into the configuration ready for fitting;
each head is movable with respect to the rod or to the clip before the device is brought into the configuration ready for fitting;
the guide orifice has an oblong shape, the greatest dimension of which extends along the length of the corresponding rail;
when projected in a plane horizontal to the spine, the relative guide trajectory, between the connection means and each of the associated vertebral assemblies, has a non-zero component;
the connection means and each of the associated vertebral assemblies are designed to slide against one another in the area of at least two respective relative guide surfaces which correspond substantially to a same spherical portion with a concavity directed toward the spine;
the relative guide trajectories, between the connection means and the two associated vertebral assemblies, are respectively centered at distinct zones.

The invention will be better understood from reading the following description which is given solely by way of example and with reference to the drawings, in which:

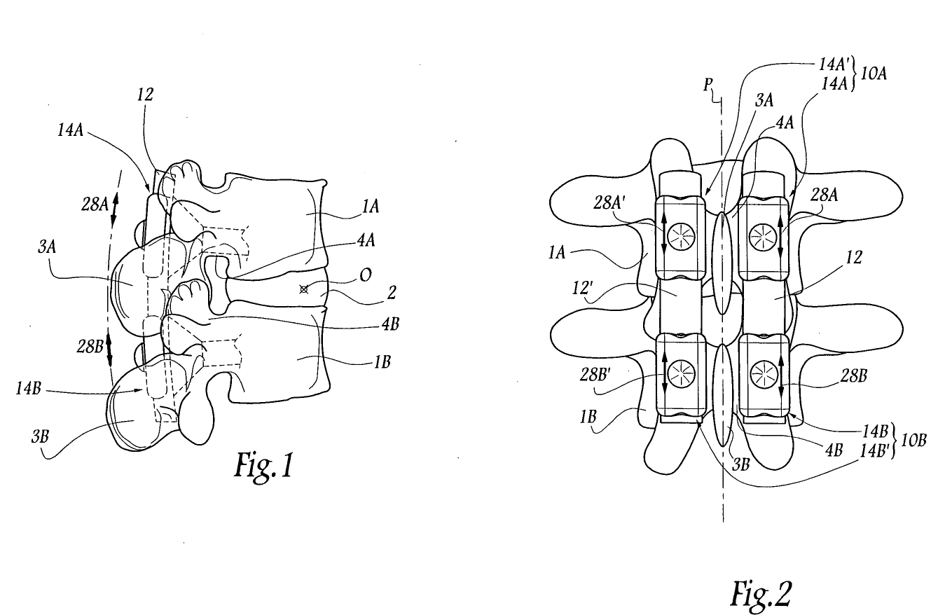

FIGS. 1 and 2 are elevation views of a first embodiment of the device according to the invention, implanted in two vertebrae, FIG. 1 corresponding to a side view of these vertebrae, while FIG. 2 corresponds to a rear view;

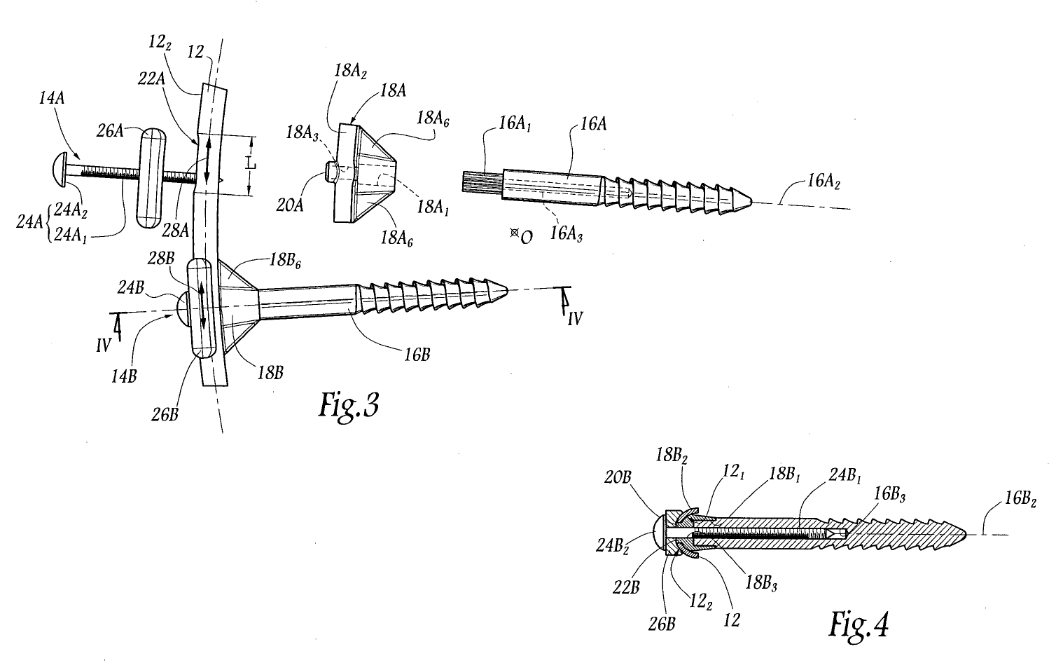

FIG. 3 is an elevation view of part of the device from FIG. 1, of which some components are represented in an exploded depiction;

FIG. 4 is a cross section along the line IV-IV in FIG. 3;

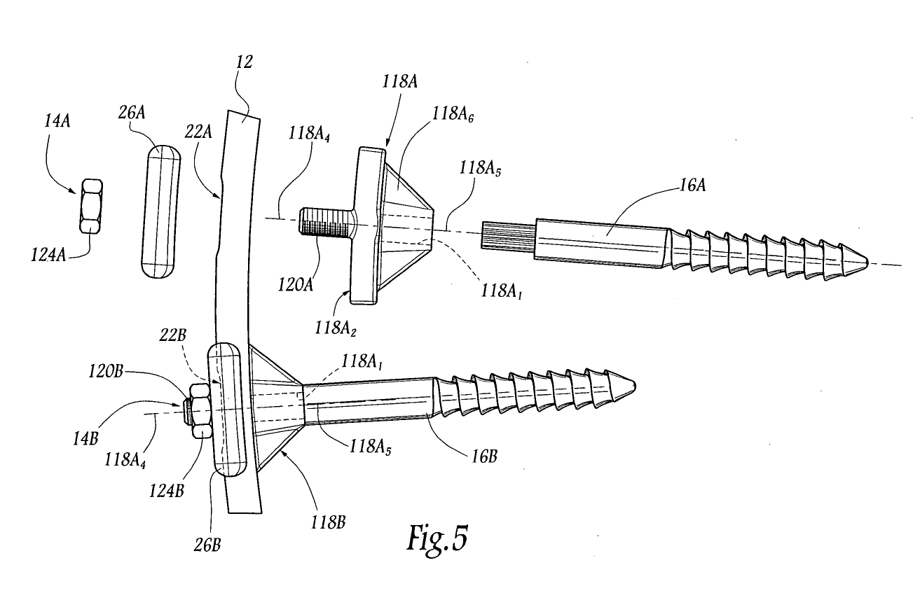

FIG. 5 is a view analogous to FIG. 3 and on an enlarged scale, illustrating a variant of the first embodiment of the device according to the invention;

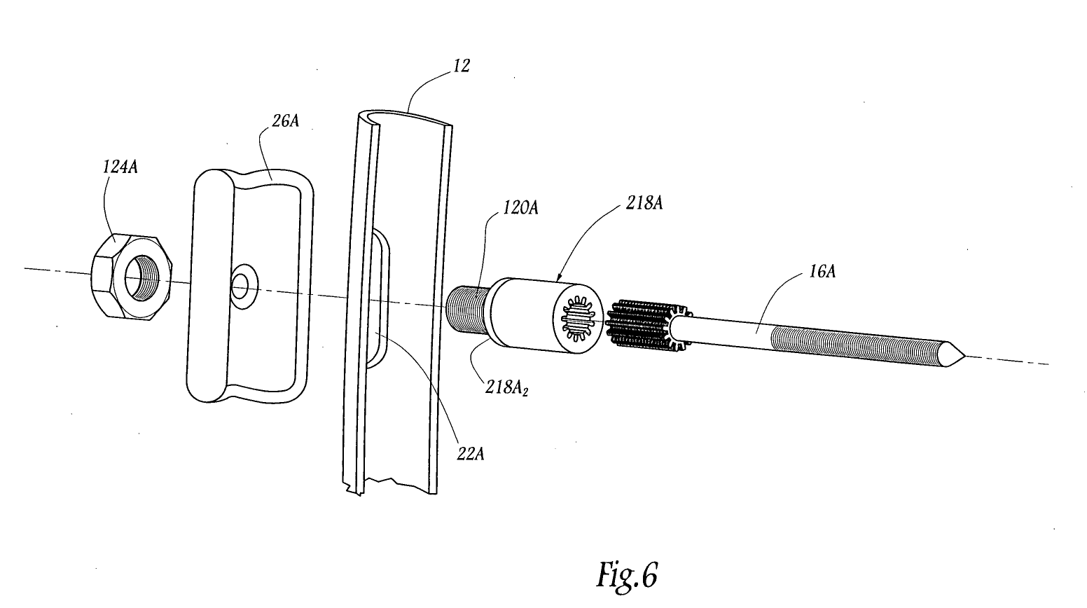

FIG. 6 is a perspective and partial view of another variant of the first embodiment of the device according to the invention;

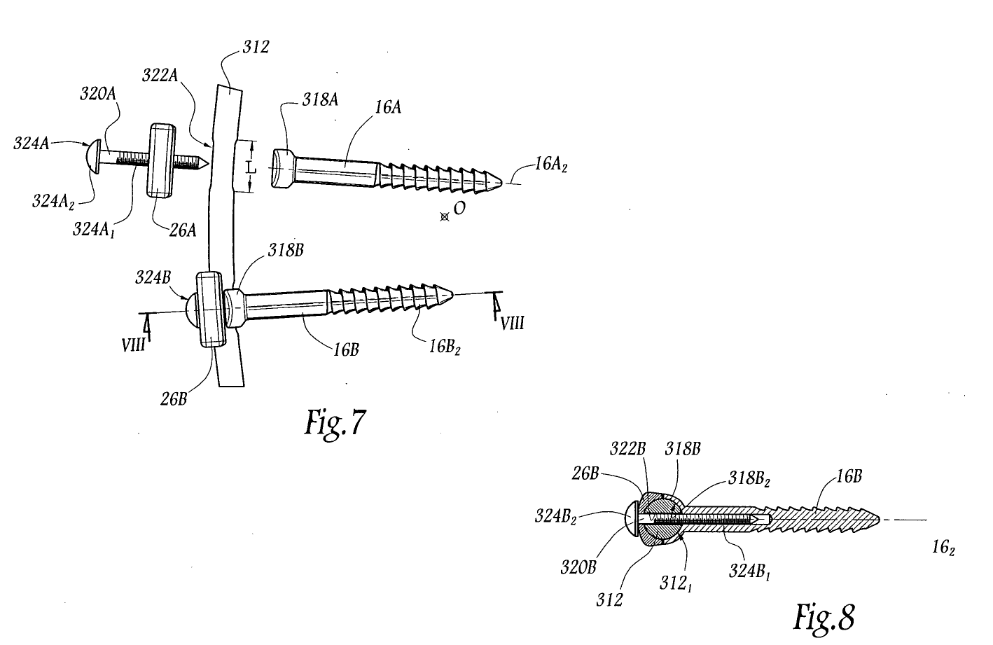

FIG. 7 is a view analogous to FIG. 3, illustrating another variant of the first embodiment of the device according to the invention;

FIG. 8 is a cross-sectional view along the line VIII-VIII in FIG. 7;

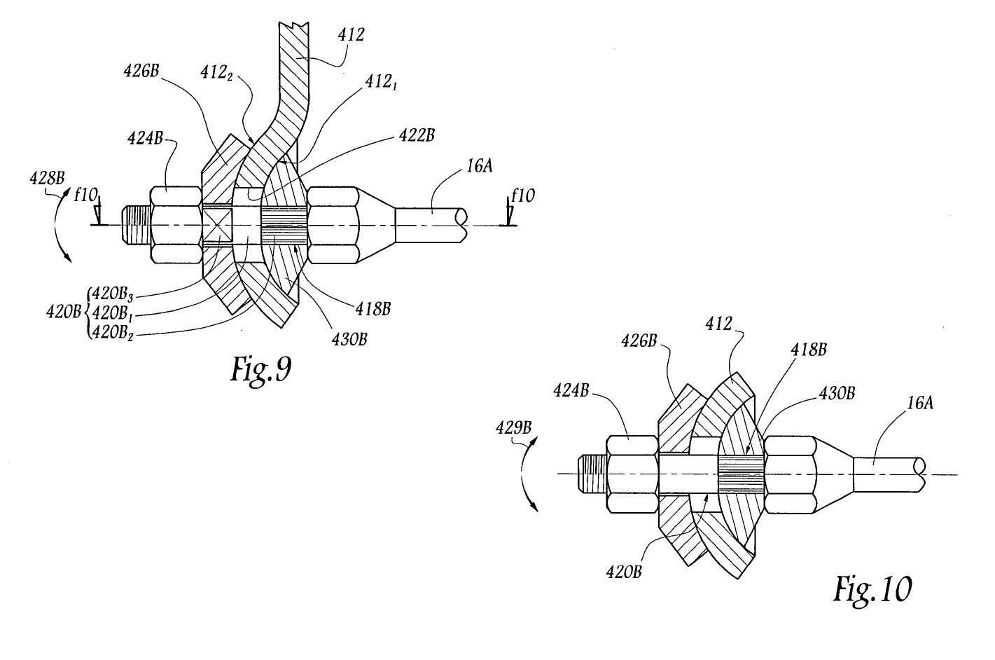

FIGS. 9 and 10 are cross sections showing another variant of the first embodiment of the device according to the invention, the sectional plane in FIG. 9 being parallel to the sagittal plane of the vertebrae, while the plane in FIG. 10 is horizontal, the plane in FIG. 10 being indicated by F₁₀-F₁₀ in FIG. 9;

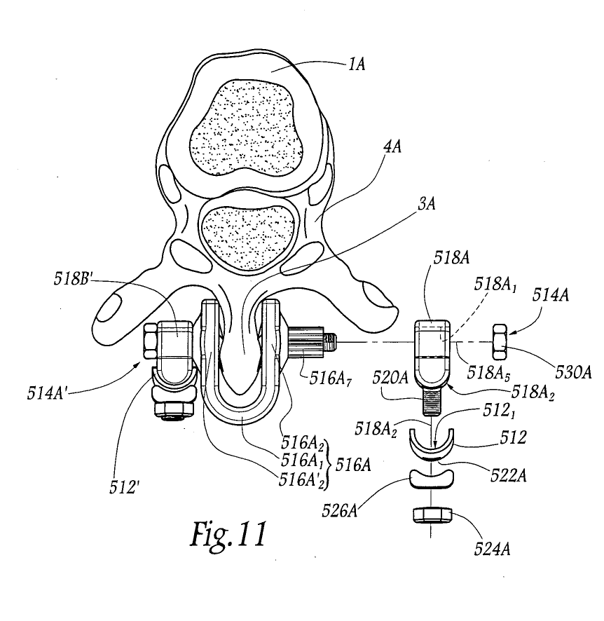

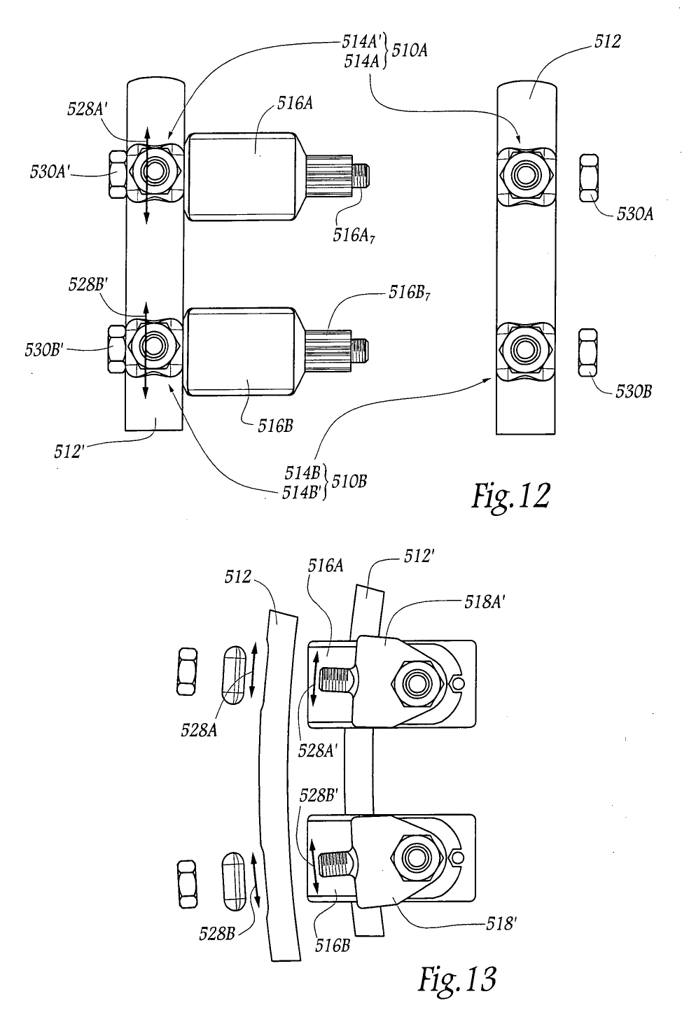

FIGS. 11 to 13 concern a second embodiment of the device according to the invention, FIG. 11 corresponding to a top view of the device implanted in a vertebra, while FIGS. 12 and 13 correspond respectively to elevation views of this device from the rear and from the side analogous to FIGS. 2 and 1, some components of the device being shown in an exploded depiction in FIGS. 11 to 13;

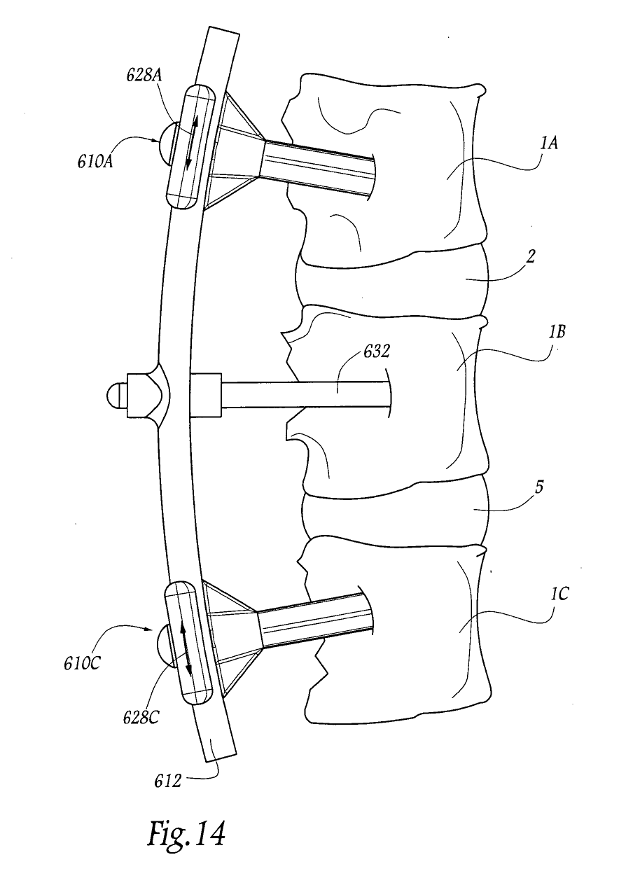

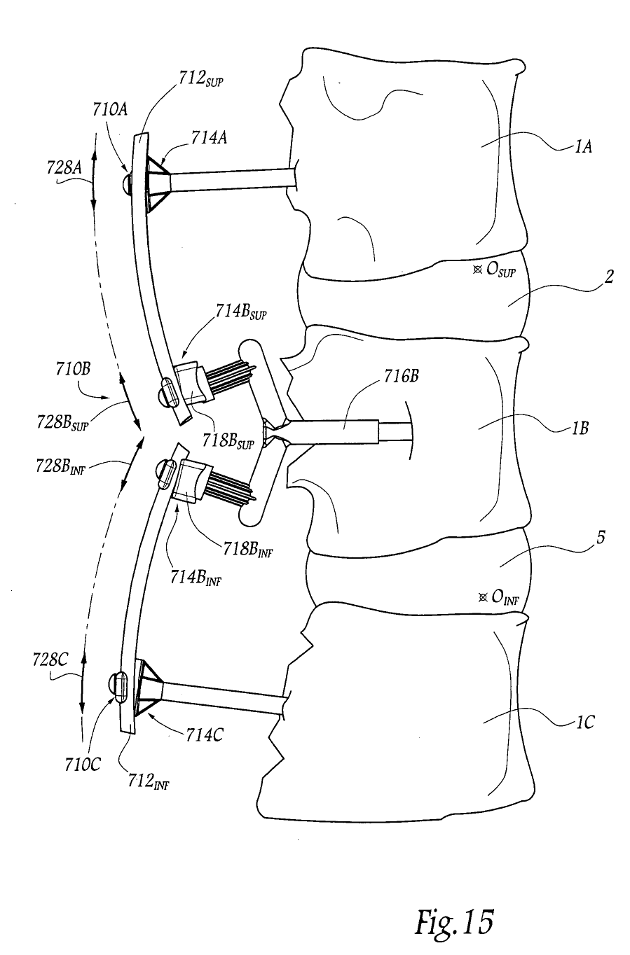

FIGS. 14 and 15 are views analogous to FIG. 1, illustrating respectively two variants of the first embodiment of the device according to the invention, implanted in three adjacent vertebrae.

FIGS. 1 and 2 show two adjacent vertebrae 1A and 1B of the lumbar spine of a human being. These vertebrae are separated from one another by an intervertebral disk 2. For clarity, the following description is based on these vertebrae in their anatomical positions, that is to say the terms “posterior”, “rear”, “anterior”, “front”, “right”, “left”, “upper”, “lower”, etc., are to be understood with respect to the spine of a patient who is standing.

In FIGS. 1 to 4, a device for stabilizing the vertebrae 1A and 1B is shown which has been implanted on the posterior aspect of the vertebrae, with a view to reproducing the articular connection between these vertebrae, while recreating the initial intervertebral space. This device basically comprises a vertebral assembly 10A implanted in the vertebra 1A, a vertebral assembly 10B implanted in the vertebra 1B, and a pair of bars 12 and 12′ connecting these assemblies to one another and extending along the spine, as described in detail below.

Each vertebral assembly 10A, 10B at the same time includes a right-hand vertebral subassembly 14A, 14B and a left-hand vertebral subassembly 14A′, 14B′, which are respectively arranged on either side of the sagittal plane P containing the spinous processes or apophysises 3A and 3B of the vertebrae 1A and 1B. The right-hand subassemblies 14A and 14B are connected mechanically by the bar 12, while the left-hand subassemblies 14A′ and 14B′ are connected mechanically by the bar 12.

Each of the subassemblies 14A, 14A′, 14B and 14B′ comprises identical components, so that, for the sake of simplicity, only the components visible in FIGS. 3 and 4 will be described in detail below, it being understood that, by convention, and for all the embodiments mentioned in the present document, the components designated by the letter “A” relate to the vertebra 1A, while the components designated by the letter “B” are associated with the vertebra 1B. Similarly, by convention, in contrast to the right-hand components, the left-hand components of the device are designated by a prime sign. It will also be noted that, overall, the right-hand and left-hand components of the device are implanted symmetrically with respect to the sagittal plane P of the spine passing through the spinous processes.

As is shown in FIGS. 3 and 4, each subassembly 14A, 14B comprises a threaded anterior rod 16A, 16B designed to fix the subassembly to the vertebrae 1A, 1B. Each rod is dimensioned to anchor itself firmly in the pedicle 4A, 4B of the vertebra, as shown in FIGS. 1 and 2.

At its posterior end, each rod 16A, 16B carries a one-piece head 18A, 18B designed to be joined rigidly to the rod. For this purpose, each head has, at its anterior end, a seat 18A₁, 18B₁ for receiving and immobilizing in rotation the posterior end 16A₁, 16B₁ of the rod, which, for example, has, in transverse section, a profile consisting of hollows and bosses complementing that of the wall of the seat. In the implantation configuration, that is to say in the configuration of the subassembly 14B in FIGS. 3 and 4, each head is totally immobilized with respect to the corresponding head. However, to facilitate the rotation of the rods about their axis 16A₂, 16B₂, in order to anchor them in the vertebral pedicles during the surgical intervention for implantation of the device, the rods are equipped with a removable head other than the head 18A, 18B, which other head (not shown) is intended to cooperate with a suitable tool for driving the rods in rotation.

On its posterior side, each head 18A, 18B is rigidly fitted with a stud 20A, 20B which projects rearward from the rest of the posterior face 18A₂, 18B₂ of the head. This stud is dimensioned so as to be received in an oblong orifice 22A, 22B which passes through the bar 12 in a generally anteroposterior direction. The orifices 22A, 22B have their greatest dimension parallel to the length of the bar 12. More precisely, the stud has an external diameter substantially equal to the width of the oblong orifice and smaller than the length of this orifice, indicated by L in FIG. 3.

As is shown in FIG. 4, the bar 12 has, in cross section, a general profile in the shape of a C, of which the recess, directed toward the front, receives the posterior end of the heads 18A and 18B. The concave and substantially semicylindrical anterior face 12₁ of the bar complements the posterior face 18A₂, 18B₂ of the heads, except at the level of the studs 20A and 20B received inside the oblong orifices 22A, 22B. In this way, each head is able to slide along the bar 12, with sliding contact of the faces 12₁ and 18A₂, or 18B₂, and guided by the cooperation of the stud and of the oblong orifice. In other words, in the area of each orifice 22A, 22B, the bar 12 forms a slide rail for the heads 18A and 18B, with a maximum relative course of length L.

To ensure that, during its use, the bar 12 cannot disengage from the studs 20A and 20B of the posterior end of the device, each subassembly 14A, 14B comprises a securing screw 24A, 24B whose rod 24A₁, 24B₁ is introduced longitudinally, from the rear of the device, into the inside of a through-hole 18A₃, 18B₃ of the head, centered on the stud 20A, 20B, and opening into the seat 18A₁, 18A₂. The head 24A₂, 24B₂ of the screw forms a rearward abutment for the bar, with the interposition of a securing cap 26A, 26B that is able to slide along the convex posterior face 12₂ of the bar 12.

Advantageously, the rod 24A₁, 24B₁ is sufficiently long to be screwed inside a complementary longitudinal orifice 16A₃, 16B₃ formed in a forward direction from the posterior end 16A₁, 16B₁ of each rod 16A, 16B. In this way, in the implantation configuration of the device, the screw 24A, 24B ensures the axial immobilization between the rod 16A, 16B and the corresponding head 18A, 18B.

In its implantation configuration, each subassembly 14A, 14B is thus connected to the bar 12 so as to be able to slide with a maximum course L. Viewed laterally, as in FIGS. 1 and 3, the slide trajectory along the spine between each subassembly and the bar is not rectilinear, but instead arched, with a center of curvature situated to the front of the bar. To do this, the bar 12 is curved inward along its length, bulging out in the rearward direction. In the example shown in FIGS. 1 to 4, the bar 12 has a lateral profile in the form of an arc of a circle, centered at a reference point O. The curvature of the bar 12 is such that, in the implantation configuration of the device, this center O is situated within the intervertebral space separating the osseous bodies of the vertebrae 1A and 1B, that is to say the space containing the disk 2, especially in the central area of this space. In this way, when the stabilizing device is implanted in the vertebrae 1A and 1B, the relative movements between these vertebrae are, at least for the most part, imposed by the guided sliding of the subassemblies 14A, 14B, 14A′ and 14B′ with respect to the connection bars 12 and 12′, the inwardly curved guide trajectories between these assemblies and these bars being designated respectively by 28A, 28B, 28A′ and 28B′. In other words, each of the trajectories 28A, 28B, 28A′ and 28B′ extends in a plane substantially parallel to the sagittal plane P and, projected in the latter, has a concavity directed toward the spine.

By virtue of their structural rigidity, the connection bars 12 and 12′ each form a guide rail for the subassemblies 14A, 14B, 14A′ or 14B′ and guarantee that the centers of curvature of these trajectories correspond to the centers of curvature of the rails that they form, which is to say that, in FIG. 1, the trajectories 28A and 28B are centered on the point O. Consequently, these trajectories are centered on the intervertebral disk space, resulting in a dynamic behavior close to the normal anatomical behavior of two adjacent vertebrae. In other words, the disk space is not reduced and disk 2 keeps a mobility substantially centered on point O.

In practice, depending in particular on the tolerances in the manufacture and fitting of the device, and because of the functional play inherent to this fitting, the relative trajectories between each subassembly and its associated bar are not necessarily centered, along their entire course, at a single point, but rather at a zone combining all the instantaneous centers of rotation between each subassembly and its bar along the maximum relative course L. In a variant not shown here, the inwardly curved profile of the bars can in some cases also be designed to impose, on the maximum relative course L, several successive instantaneous centers of rotation.

To guarantee a homogeneous dynamic behavior between the vertebrae 1A and 1B along the entire course of the trajectories 28A, 28B, 28A′ and 28B′, the connection bars 12 and 12′ are implanted substantially parallel to one another, in an overall vertical direction with respect to the spine of a patient who is standing.

FIGS. 5, 6, 7-8 and 9-10 show, respectively, four different variants of the stabilizing device from FIGS. 1 to 4. By convention, the identical elements between these variants and the device in FIGS. 1 to 4 have been given the same reference numbers as those used above.

The device in FIG. 5 differs from that of FIGS. 1 to 4 in terms of the heads of each of its vertebral assemblies. Instead of the stud 20A, 20B inside which a securing screw is introduced, the stud 120A, 120B of each head 118A, 118B is solid and extends rearward, from the posterior face 118A₂ of the head, by a sufficient length ensuring that a nut 124A, 124B can be screwed around its posterior end, the outer face of the stud being threaded for this purpose. In the implantation configuration, this nut holds the stud through the corresponding oblong orifice 22A, 22B of the connection bar 12, with the same freedom of mutual sliding as for the device in FIGS. 1 to 4.

As the securing screw 24A, 24B is replaced by the nut 124A, 124B, a pin (not shown) or any other suitable mechanical means is used to axially immobilize the head 118A, 118B relative to its anchoring rod 16A, 16B. The advantage of this variant lies in the possibility of providing the surgeon with a set of several heads 118A, 118B whose respective main axes 118A₄, 118B₄, that is to say the respective longitudinal axes of the corresponding studs 120A, 120B, are inclined with different respective angles relative to the longitudinal axis 118A₅, 118B₅ of the seat 118A₁, 118B₁ for attachment to the rod 16A, 16B. In this way, once the surgeon has anchored the rod 16A, 16B, he chooses one of the heads from among the set available to him and thus adjusts the longitudinal orientation of the stud 120A, 120B of the implanted device relative to the anchoring rod. This adjustment makes it possible, in particular, to render the axis 118A₄, 118B₄ of the stud of the implanted head substantially perpendicular with the direction tangential to the bar 12 in the area of the orifice 22A, 22B for receiving this stud, which thus facilitates the relative sliding movement between each vertebral subassembly and the bar.

The variant in FIG. 6 differs from that in FIG. 5 in terms of the shape of its heads, of which only the head 218A is visible in FIG. 6, which illustrates the multiplicity of geometries of the possible heads for the device according to the invention. This head 218A is cylindrical with circular external cross section and is particularly compact compared to the head 118A of FIG. 5, while the head 118A proves, during use, to be stronger than the head 218A, on the one hand because of its posterior face 118A₂ being longer than the posterior face 218A₂ of the head 218A and, on the other hand, because of the presence of upper and lower reinforcements 118A₆. Similar reinforcements 18A₆, 18B₆ are also present in the device in FIGS. 1 to 4.

The variant embodiment of the device in FIGS. 7 and 8 differs from the device of FIGS. 1 to 4 in terms of the contour of the cross section of the connection bars connecting the vertebral subassemblies. As is shown in FIGS. 7 and 8, each connection bar or rail 312 thus has a cross section of substantially circular shape. Each bar generally forms an arched rod, centered at a point analogous to the point O for the bars 12 and 12′ of the device in FIGS. 1 to 4. The round cross section of the bar 312 induces specific features as regards the components of each vertebral subassembly connected slidably to this bar. More precisely, in the example in FIGS. 7 and 8, each bone-anchoring rod 16A, 16B is made integral, at its posterior end, with a head 318A, 318B of semicylinder shape with a circular base and a longitudinal axis which is substantially perpendicular to the axis 16A₂, 16B₂ of the rod and substantially parallel to the bar 312. The concave posterior face 318A₂ of each of these heads is complementary to the anterior face 312₁ of the bar 312 and forms, with the latter, a sliding contact.

To guarantee the guidance of this inwardly curved sliding, and to limit the maximum course of this sliding, the bar 312 is traversed, in a generally anteroposterior direction, by two separate orifices 322A, 322B distributed along the length of the bar. In cross section, each of these orifices has an oblong section, of length L, the greatest dimension of which is parallel to the length of the bar 312. In addition, each vertebral subassembly comprises a securing screw 324A, 324B, of which the posterior end part 320A, 320B of the rod forms a sliding stud introduced longitudinally into the corresponding orifice 422A, 422B in a manner analogous to the stud of the device in FIGS. 1 to 4.

Moreover, in a manner substantially analogous to the securing screws 24A, 24B of the device in FIGS. 1 to 4, each screw 324A, 324B comprises, on the one hand, a distal rod part 324A₁, 324B₁ screwed inside the anchoring rod 16A, 16B, and, on the other hand, a head 324A₂, 324B₂ for holding a cap 26A, 26B mounted slidably on the posterior face 312₂ of the bar or rail 312.

The variant embodiment in FIGS. 9 and 10 differs from the devices of FIGS. 1 to 8 in terms of a greater freedom of relative movement between each vertebral subassembly and its associated guide bar. Rather than having oblong orifices for receiving the head of each subassembly, the bar 412 shown only in part in FIGS. 9 and 10 is traversed, in an anteroposterior direction, by an orifice 422B of substantially circular cross section which receives the head 418B of the subsassembly visible in the figures, another orifice of substantially circular cross section being provided in the end part of the bar remote from that shown. For clarity, only the subsassembly visible in FIGS. 9 and 10 will be described in detail below, it being understood that, as for the devices described above, the other subassemblies of the device have similar arrangements.

The head 418B is designed, in its anterior end part, so that it can be attached to the bone-anchoring rod 16A in the same way as described above for the devices in FIGS. 1 to 8. In its posterior end part, the rod 418B forms a stud 420B whose substantially cylindrical part 420B₁ has a diameter much smaller than that of the orifice 422B in which this part 420B₁ is housed in the implantation configuration of the device. The anterior end 420B₂ of the stud is attached to an anterior washer 430B, for example by cooperation of matching hollows and bosses provided on the stud and washer. Likewise, the posterior end 420B₃ of the stud 420B is integral with a posterior washer 426B. This washer 426B serves as a cap for securing the device, and a retention nut 424B, functionally analogous to the nut 124B of the device in FIGS. 5 and 6, is provided at the posterior end of the stud.

When the device in FIGS. 9 and 10 is implanted on the spine, the subassembly shown in the figures is able to move relative to the bar 412 by virtue of the peripheral spacing between the stud 420B and the wall of the orifice 422B. Looking at the device laterally with respect to the spine, as in FIG. 9, a sliding movement, generally parallel to the longitudinal direction of the spine, is permitted between the subassembly and the bar, this movement being analogous to the one corresponding to the trajectory 28B in FIGS. 1 and 3. In other words, the projection of the displacement trajectory on the sagittal plane P of the spine, designated as 428B in FIG. 9, and constituting the sagittal component of this trajectory, is inwardly curved by bulging outward toward the rear. In addition to this sagittal component, the trajectory has a transverse non-zero component, corresponding to the projection of the trajectory in a plane horizontal with respect to the spine of a patient who is standing. It will be noted that, for the embodiments in FIGS. 1 to 8, the guide trajectories 28A, 28B, 28A′, 28B′ do not have a sagittal component, except for functional play. By virtue of this transverse component, designated as 429B in FIG. 10, the device in FIGS. 9 and 10 has an internal clearance transverse to the longitudinal direction of the spine, ensuring greater comfort for the patient during combined movements of torsion and of flexion/extension of the spine.

It will be appreciated that, for this purpose, the washers 426B and 430B are respectively designed to slide against the anterior face 412₁ and posterior face 412₂ of the bar 412, in such a way as to guide in an inwardly curved manner the clearance movements between the head 418B and the bar, without impeding them.

In practice, the posterior surface 430B₁ of the washer 430B and the cooperating surface delimited by the anterior face 412₁ of the bar 412 correspond substantially to the same sphere portion, of which the concavity is directed toward the spine. The same advantageously applies to the anterior surface 426B₁ of the washer 426B and the cooperating surface delimited by the posterior face 412₂.

FIGS. 11 to 13 show a second embodiment of the device for stabilizing the spine. As for the devices in FIGS. 1 to 8, the device in FIGS. 11 to 13 basically comprises a vertebral assembly 510A intended to be implanted in the vertebra 1A, a vertebral assembly 510B intended to be implanted in the vertebra 1B, and two bars or rails 512, 512′ connecting these two assemblies to one another. Each vertebral assembly 510A, 510B comprises a right-hand vertebral subassembly 514A, 514B and a left-hand vertebral subassembly 514A′, 514B′, the bar 512 connecting the right-hand subassemblies, while the bar 512′ connects the left-hand subassemblies.

This second embodiment differs from the device of FIG. 5 basically in terms of the bone-fixation zone on the vertebrae 1A and 1B. Rather than having a pedicle fixation, each assembly 510A, 510B comprises a clip 516A, 516B intended to enclose, from the rear, the spinous process 3A, 3B of each vertebra, as shown in FIG. 11. Each clip is common to both vertebral subassemblies of the assembly in question, which reduces the number of components of the device compared to those in FIGS. 1 to 10.

Each clip 516A, 516B has in cross section, that is to say in a sectional plane substantially vertical when this clip is engaged on its apophysis 3A, 3B, a profile generally in the shape of a U, of which the base 516A₁ is directed toward the rear, while the two wings, namely the right-hand wing 516A₂ and the left-hand wing 516A₂, are arranged laterally on either side of the apophysis. To improve the mechanical hold of the fixation of each clip, the mutually opposing faces of the branches have a raised and hollowed relief designed to grasp the bone substance of the apophysis.

Each vertebral assembly 510A, 510B also comprises components associated with the right-hand and left-hand sides of the apophysis 3A, 3B, only the components of the subassembly 514A being described in detail below, it being understood that the other subassemblies 514B, 514A′ and 514B′ comprise analogous components, with the conventions described above regarding the reference numbers.

The subassembly 514A comprises a solid head 518A having a substantially semicylindrical convex posterior face 518A₂ and intended to slide along the bar 512 of C-shaped cross section, against its anterior face 512₁. On its posterior side, this head has a stud 520A similar to the stud 120A of the device in FIG. 5 and received in an oblong orifice 522A passing right through the bar 512, and of which the main axis is parallel to the length of the bar 512. This stud is associated with a nut 524A and with a securing cap 526A which are analogous to the nut 124A and to the cap 26A. In addition, the head 518A is fixed securely to the corresponding wing 516A₂ of the clip 516A by this head being fitted to a corresponding support cylinder 516A₇ formed integrally with this wing. More precisely, the head has a through-orifice 518A₁ designed to be engaged around the support cylinder 516A₇, an additional nut 530A being attached on the side of the head opposite from the wing, by being screwed onto the corresponding threaded end of the support cylinder, in order to immobilize the head on the axis 518A₅ of this seat.

Advantageously, the outer face of the support cylinder 516A₇ and the wall of the seat 518A₁ are designed to make it possible to adjust the angular position of the head relative to this cylinder, around the axis 518A₅, before the nut 530A is securely tightened. In this way, when the device is in the process of being implanted, the surgeon is able to adjust the position of the head 518A relative to the clip 516A by driving this head in rotation about the axis 518A₅, particularly with a view to rendering the longitudinal axis 518A₄ of the stud 520A substantially perpendicular to the direction tangential to the bar 512 in the area of its receiving orifice 522A. In other words, before the device is fixed in its implantation configuration, the device in FIGS. 11 to 13 allows the longitudinal direction of the sliding stud of each head to be adjusted relative to the components of the device that are firmly fixed to the vertebrae.

When in use, the device in FIGS. 11 to 13 behaves in a manner identical to that of FIGS. 1 to 4, since each bar 512, 512′ is inwardly curved in a similar way to the bars 12 and 12′, by being arranged parallel to one another and on either side of the apophyses 3A and 3B. The inwardly curved guide trajectories between each subassembly 514A, 514B, 514A′, 514B′ and the bars 512, 512′, respectively designated by 528A, 528B, 528A′, 528B′, are centered at a point O.

FIGS. 14 and 15 show variants of the device for intervertebral stabilization, intended to be implanted in three adjacent vertebrae 1A, 1B and 1C that are separated by disks 2 and 5. For the sake of clarity, only the vertebral bodies of these vertebrae are shown, and in a schematic manner.

The device in FIG. 14 corresponds to a certain extent to the device in FIGS. 1 to 4, with longer connection bars, of which the median part is anchored in the pedicle of the intermediate vertebra B. More precisely, this device comprises two vertebral assemblies 610A, 610C identical respectively to the vertebral assemblies 10A and 10B of FIGS. 1 to 4, and a pair of left-hand and right-hand connection bars which are identical to one another and connect these two assemblies, only the right-hand bar 612 being visible in FIG. 12 and being described below. The bar or rail 612 has the same properties as the bar 12 from FIGS. 1 to 4 in terms of its sliding connection and relative guiding with the vertebral assemblies 610A and 610C. The corresponding guide trajectories are designated as 628A and 628C in FIG. 12. The center of curvature of the rail 612, designated by O, is thus situated vertically in the area of the intermediate vertebra 1B, in front of the latter, so that these three vertebrae are given overall freedoms of movement analogous to those of the two adjacent vertebrae 1A and 1B in FIGS. 1 to 4.

To reinforce the mechanical stability of the device from FIG. 12, the median part of the bar 612 is provided with a broach 632 for anchoring in the pedicle of the vertebra B. This broach is connected rigidly to the rail formed by the bar 612.

FIG. 15 shows another stabilizing device intended to be implanted in three adjacent vertebrae 1A, 1B and 1C. This device corresponds to a certain extent to the juxtaposition of two devices from FIGS. 1 to 4. More precisely, this device comprises three vertebral assemblies 710A, 710B and 710C anchored in the pedicles of the vertebrae 1A, 1B and 1C. A pair of inwardly curved bars connects the assemblies 710A and 710B in the same way as the bars 12 and 12′ connect the assemblies 10A and 10B in FIGS. 1 to 4, while another pair of bars connects the assemblies 710B and 710C, also in the same way as the bars 12 and 12′ connect the assemblies 10A and 10B in FIGS. 1 to 4. In FIG. 13, which illustrates the right-hand side of the device, only one bar 712_{SUP} connecting the assemblies 710A and 710B and one bar 712_{INF} connecting the assemblies 710B and 710C are shown. The assemblies 710A and 710B are connected slidably to the bar 712_{SUP}, on inwardly curved relative guide trajectories, designated as 728A, 728B_{SUP} and centered at a point O_{SUP}, while the assembly 710D and 710C are connected to the bar 712_{INF} so as to slide on inwardly curved guide trajectories 728B_{INF} and 728C that are centered at a point O_{INF}. The center O_{SUP} is situated in the intervertebral space occupied by the disk 2, while the center O_{INF} is situated in the intervertebral space occupied by the disk 5.

For the sake of clarity, only the right-hand side of this device, visible in FIG. 15, is described in detail below, it being understood that analogous features are provided on the left-hand side of the device, in a manner substantially symmetrical to a sagittal plane passing through the spinous processes of the vertebrae. Thus, the right-hand subassembly 710B comprises both an upper subassembly 714B_{SUP} and a lower subassembly 714B_{INF}, both of them supported by the same pedicle-anchoring rod 716B. Each of these subassemblies comprises a head 718A_{SUP}, 718A_{INF} substantially analogous to the head of each subassembly of the device in FIG. 6. The right-hand subassemblies 714A and 714C are for their part analogous to the subassemblies 14A and 14B in FIGS. 1 to 4.

When in use, the device in FIG. 15 ensures kinematics appropriate to each pair of vertebrae 1A/1B and 1B/1C respectively analogous to the kinematics described in detail for vertebrae 1A/1B in FIGS. 1 to 4.

A number of modifications and variants of the stabilizing devices described above are also conceivable:
the shapes of the vertebral subassemblies of the devices for three vertebrae are not limited to those represented in FIGS. 14 and 15, and instead these subassemblies can equally have the subassembly forms envisaged in FIGS. 1 to 13;
the bars or rails for sliding connection between the vertebral subassemblies are not necessarily intended to be implanted in the posterior face of the vertebrae; bars or rails that are laterally offset to the right or left of the vertebrae, or are arranged on the anterior side of the vertebrae, are conceivable; in the case of rails provided on the anterior side, these rails are preferably supported by a common component, in particular a plate, which is easier to fit in place than two independent bars;
in all the embodiments envisaged in the figures, the connection bars have a continuous curvature along their entire length, such that the relative slide trajectories between each vertebral subassembly and this bar are centered at a single point, or at least in a single zone; it is possible to design each bar with different curvatures in the area of its oblong guide orifices for sliding of each subassembly, so that, for a given bar, the two sliding trajectories associated respectively with each vertebral subassembly are then centered at two points, or at least in two zones, distinct from one another, both of these two points being nonetheless situated within the interosseous space delimited between the two vertebrae to which the vertebral assemblies are fixed;
the median part of each bar or rail, connecting the two end parts of the rail along which the vertebral subassemblies slide, can have a rectilinear structure or other structure, since this has no influence on the curvature of the relative guide trajectories; and/or
the vertebrae can be fitted with a device on just one side; in this case, each vertebral assembly comprises only one subassembly.

1. Device for dynamically stabilizing the spine intended to reproduce an intervertebral articular connection, comprising at least two vertebral assemblies designed to be each fixed respectively to the bone a vertebra from among at least two different vertebrae of the spine, said device additionally comprising rigid means for connection between the two vertebral assemblies or between two of said vertebral assemblies, wherein said rigid means and said vertebral assemblies are designed such that, when the device is in implantation configuration, they are adapted to be connected to one another so as to slide along a relative guide trajectory which, projected in the sagittal plane of the spine, is curved along the spine, having a concavity directed toward the spine and being centered at a zone contained within the interosseous space delimited between the two vertebrae to which the two assemblies are fixed.

2. Device according to claim 1, wherein the vertebral assemblies are respectively designed to be fixed to two adjacent vertebrae, and in that the projection, in the sagittal plane of the spine, of the relative guide trajectory between the connection means and each of the two associated vertebral assemblies, is centered at a zone contained within the disk space separating the two vertebrae to which the two assemblies are fixed.

3. Device according to claim 1, wherein each vertebral assembly comprises two subassemblies that can be fixed to the same vertebra, on either side of its spinous process.

4. Device according to claim 1, wherein the connection means for the two assemblies comprise two curved rigid rails for guiding the vertebral assemblies, which rails are substantially parallel to one another and along which, respectively, opposite lateral parts of each vertebral assembly are designed to slide along said trajectory when the device is in implantation configuration.

5. Device according to claim 4, wherein the two rails are designed to extend along and on either side of the spinous processes of the vertebrae.

6. Device according to claim 4, wherein the two rails are supported by one and the same component designed to extend, in the longitudinal direction of the rails, along the anterior side of the vertebrae.

7. Device according to claim 4, wherein each lateral part of each vertebral assembly comprises a head for sliding along the corresponding rail, this head being equipped with a stud received in a guide orifice delimited by the rail.

8. Device according to claim 4, wherein each lateral part of each vertebral assembly comprises a pedicle-anchoring rod or a clip for fastening on the process.

9. Device according to claim 7, wherein each lateral part of each vertebral assembly comprises a pedicle-anchoring rod or a clip for fastening on the process and wherein the longitudinal direction of the stud of each head is adjustable relative to the rod or to the clip before bringing the device into the configuration ready for fitting.

10. Device according to claim 7, wherein each lateral part of each vertebral assembly comprises a pedicle-anchoring rod or a clip for fastening on the process and wherein each head is movable with respect to the rod or to the clip before bringing the device into a configuration ready for fitting.

11. Device according to claim 7, wherein said guide orifice has an oblong shape, the greatest dimension of which extends along the length of the corresponding rail.

12. Device according to claim 1, wherein, when projected in a plane horizontal to the spine, the relative guide trajectory, between the connection means and each of the associated vertebral assemblies, has a non-zero component.

13. Device according to claim 12, wherein the connection means and each of the associated vertebral assemblies are designed to slide against one another in the area of at least two respective relative guide surfaces which correspond substantially to a same sphere portion with a concavity directed toward the spine.

14. Device according to claim 1, wherein the relative guide trajectories, between the connection means and the two associated vertebral assemblies, are respectively centered in distinct zones.
